(19) United States
(12) Patent Application Publication
(10) Pub. No.: US 2022/0000707 A1
(43) Pub. Date: Jan. 6, 2022

(54) SKIN CARE DEVICE

(71) Applicant: LG ELECTRONICS INC., Seoul (KR)

(72) Inventors: Nayoung KIM, Seoul (KR); Gueisam LIM, Seoul (KR); Yongju YANG, Seoul (KR); Munseong KANG, Seoul (KR); Dongwon KIM, Seoul (KR); Sangwon KIM, Seoul (KR); Saejung KIM, Seoul (KR)

(73) Assignee: LG ELECTRONICS INC., Seoul (KR)

(21) Appl. No.: 17/432,783

(22) PCT Filed: Feb. 21, 2020
(86) PCT No.: PCT/KR2020/002561

§ 371 Date: Aug. 20, 2021

Related U.S. Application Data
(60) Provisional application No. 62/809,378, filed on Feb. 22, 2019.

(51) Int. Cl. A61H 23/02; A61H 7/00

(52) U.S. Cl. CPC A61H 23/0245; A61H 2201/1207; A61H 7/004

(57) ABSTRACT

Discussed is a skin care device comprising: a main body having a processor, and an ultrasonic vibrator assembly disposed on one end of the main body and forming a contact surface with a skin of a user, wherein the processor controls the ultrasonic vibrator assembly to apply an ultrasonic vibration to the skin according to at least one of a frequency characteristic, an output characteristic, and a duty ratio characteristic for removing wastes on a surface of the skin.

### TECHNICAL FIELD

The present disclosure relates to a skin care device.

### BACKGROUND ART

Skin care aims at maintaining clean, soft skin without blemishes, and in particular, the most interest is formed in skin care of the face among body parts. Therefore, people want to keep their skin clean by receiving a massage, applying a functional cosmetic product, or using various cleaning products for facial skin care.

Among them, the importance of washing the face to remove wastes from the skin is gradually increasing, and for washing the face, people apply a cleansing product to their face by hand and then wash them with water to remove wastes from the skin.

However, when washing your face using your hands, since the cleaning products may not be delivered evenly to the skin and bacterial infection may occur by the hands, recently, the method of indirectly applying the cleaning products to the face using various tools is being used. In particular, skin care devices that, among these tools, includes a brush and a vibration motor and cleans the skin through vibration of the brush or applies ultrasonic vibration to the skin to clean the skin have emerged.

### DISCLOSURE

### Technical Problem

An object to be solved by the present disclosure is to provide a skin care device that maximizes cleaning power against wastes present on the skin surface.

### Technical Solution

The skin care device according to an embodiment of the present disclosure may be implemented to apply ultrasonic vibrations having a frequency characteristic, an output characteristic, and a duty ratio characteristic for maximizing the cleaning power for wastes on the skin surface to the skin.

According to an embodiment, the frequency of the ultrasonic vibration applied to the skin may be less than 1 MHz.

According to an embodiment, the frequency of the ultrasonic vibration applied to the skin may be 0.13 MHz or more and less than 1 MHz.

According to an embodiment, the frequency of the ultrasonic vibration applied to the skin may be set closer to 0.35 MHz than 0.13 MHz and 1 MHz.

According to an embodiment, the output of the ultrasonic vibration applied to the skin may be set closer to 70 mW/cm² than to 25 mW/cm² and 115 mW/cm².

According to an embodiment, the duty ratio of the ultrasonic vibration applied to the skin may have a range of 50% or more and less than 70%.

According to an embodiment, the duty ratio of the ultrasonic vibration applied to the skin may be set closer to 60% than the 50% and 70%.

A skin care device according to an embodiment of the present disclosure may include a brush having a plurality of protrusions that come into contact with the skin, in which the plurality of protrusions may be arranged in a Fibonacci spiral pattern.

According to an embodiment, the thickness or height of the plurality of protrusions may increase from the center toward the outside of the brush.

According to an embodiment, the plurality of protrusions may be implemented with silicon having a hardness of 30 or more and less than 50.

According to an embodiment, the hardness of the plurality of protrusions may be closer to 40 than 30 and 50.

A skin care device according to an embodiment of the present disclosure may include an ultrasonic vibrator assembly that forms a contact surface with the skin, and a brush including a plurality of protrusions that form a contact surface with the skin.

### Advantageous Effect

According to an embodiment of the present disclosure, the skin care device can more effectively remove wastes existing on the skin surface by applying ultrasonic vibrations to the skin based on frequency characteristics, output characteristics, and duty ratio characteristics for maximizing cleaning power.

In addition, the skin care device is provided with a silicone brush having an array pattern, a thickness pattern, and hardness for maximizing cleaning power, so that wastes existing on the skin surface can be more effectively removed.

In addition, the skin care device may provide improved cleaning power compared to hand cleansing or a conventional cleansing device by applying both ultrasonic vibration and brush micro-vibration to the skin. Accordingly, it is possible to improve the skin health of the user and provide high satisfaction with the product.

### DESCRIPTION OF DRAWINGS

FIG. 1 is a perspective view illustrating a skin care device according to an embodiment of the present disclosure.

FIG. 2 is a perspective view illustrating a package including a skin care device and a cradle illustrated in FIG. 1.

FIG. 3 is an exploded view illustrating the skin care device illustrated in FIG. 1.

FIG. 4 is a cross-sectional view for explaining the structure of the ultrasonic vibrator assembly illustrated in FIG. 3.

FIG. 5 is an example of experimental data obtained by measuring the difference in cleaning power according to a change in the frequency and output of ultrasonic vibration.

FIGS. 6 to 8 are examples of experimental data obtained by measuring the difference in the cleansing region, the difference in the residual region of the waste mimetic body, and the difference in skin brightness according to the change in the frequency and output of ultrasonic vibration.

FIG. 9 is an example of experimental data obtained by measuring a difference in cleaning power according to a change in a duty ratio of ultrasonic vibrations.

FIGS. 10 to 12 are examples of experimental data obtained by measuring the difference in the cleansing region according to the change in the duty ratio of ultrasonic vibration, the difference in the residual region of the waste mimetic body, and the difference in skin brightness.

FIG. 13 is an example of experimental data obtained by measuring a difference in cleaning power according to a change in a duty ratio in an intermittent mode of ultrasonic vibration.

FIGS. 14 to 16 are examples of experimental data obtained by measuring the difference in the cleansing region, the difference in the residual region of the waste mimetic body, and the difference in skin brightness according to a change of the duty ratio in an intermittent mode of ultrasonic vibration.

FIG. 17 is a diagram illustrating a frequency range of ultrasonic vibration for maximizing cleaning power of a skin care device according to an embodiment of the present disclosure.

FIG. 18 is a view for explaining a brush that vibrates finely by driving a vibration motor illustrated in FIG. 3.

FIG. 19 is an example of experimental data obtained by measuring the difference in cleaning power according to the protrusion shape and pattern of the brush.

FIGS. 20 to 22 are examples of experimental data obtained by measuring the difference in the cleansing region, the difference in the residual region of the waste mimetic body, and the difference in skin brightness according to the protrusion shape and pattern of the brush.

FIG. 23 is an example of experimental data obtained by measuring a difference in cleaning power according to a change in thickness of a plurality of protrusions arranged in a Fibonacci spiral pattern of a brush.

FIGS. 24 to 26 are examples of experimental data obtained by measuring the difference in the cleansing region, the difference in the residual region of the waste mimetic body, and the difference in skin brightness according to the change in the thickness of the plurality of protrusions arranged in the Fibonacci spiral pattern of the brush.

FIG. 27 is an example of experimental data obtained by measuring a difference in cleaning power according to hardness of a plurality of protrusions of a brush.

FIGS. 28 to 30 are examples of experimental data obtained by measuring the difference in the cleansing region, the difference in the residual region of the waste mimetic body, and the difference in skin brightness according to the hardness of the plurality of protrusions of the brush.

FIG. 31 is an example of experimental data obtained by measuring the difference in cleaning power according to the hardness and surface coating of a plurality of protrusions of the brush.

FIGS. 32 to 34 are examples of experimental data obtained by measuring the difference in the cleansing region, the difference in the residual region of the waste mimetic body, and the difference in skin brightness according to the hardness and surface coating of the plurality of protrusions of the brush.

FIG. 35 is an example of experimental data obtained by measuring a difference in cleaning power according to whether a combination of ultrasonic vibration and brush micro-vibration is applied.

FIGS. 36 to 38 are experimental data obtained by measuring the difference in the cleansing region, the difference in the residual region of the waste mimetic body, and the difference in skin brightness according to whether the combination of ultrasonic vibration and brush micro-vibration is applied.

FIG. 39 is an exemplary view illustrating the difference in cleaning power when only one of ultrasonic vibration and brush micro-vibration is applied and when the combination of ultrasonic vibration and brush micro-vibration is applied.

### BEST MODE

Hereinafter, the embodiments disclosed in the present specification will be described in detail with reference to the accompanying drawings, but the same or similar components are assigned the same reference numerals regardless of reference numerals, and overlapping descriptions thereof will be omitted. The suffixes “module” and “part” for the components used in the following description are given or mixed in consideration of only the ease of writing the specification, and do not have distinct meanings or roles by themselves. In addition, in describing the embodiments disclosed in the present specification, if it is determined that detailed descriptions of related known technologies may obscure the subject matters of the embodiments disclosed in the present specification, the detailed description thereof will be omitted. In addition, it should be understood that the accompanying drawings are only for easy understanding of the embodiments disclosed in the present specification, and the technical spirit disclosed herein is not limited by the accompanying drawings, and all changes, equivalents, and substitutes included in the spirit and the technical scope of the present disclosure are included.

Terms including an ordinal number, such as first and second, may be used to describe various components, but the components are not limited by the terms. The above terms are used only for the purpose of distinguishing one component from another.

When a component is referred to as being “connected” or “accessed” to another component, it should be understood that the component may be directly connected or accessed to another component, but there may be other components in between. On the other hand, when it is said that a component is “directly connected” or “directly accessed” to another element, it should be understood that there are no other component in between.

The singular expression includes the plural expression unless the context clearly dictates otherwise.

It should be understood that, in the present application, terms such as “comprises” and “have” are intended to designate that a feature, number, step, operation, component, part, or combination thereof described in the specification exists, but this does not preclude the possibility of the existence or addition of one or more other features, numbers, steps, operations, components, parts, or combinations thereof.

Hereinafter, embodiments of the present disclosure will be described in detail with reference to the accompanying drawings in the present specification.

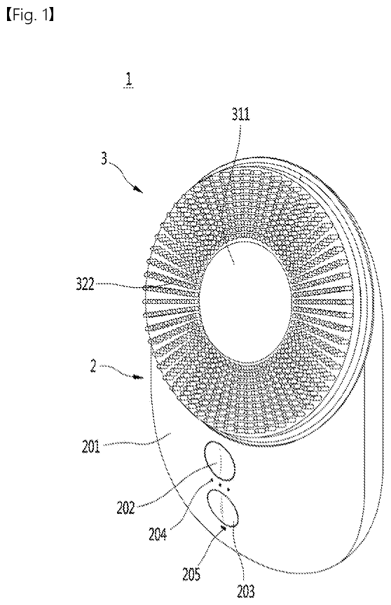

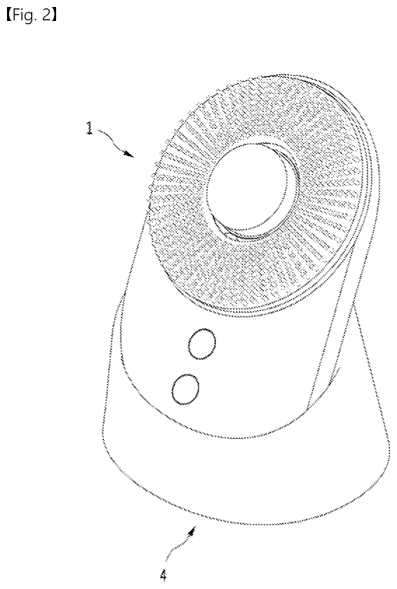

FIG. 1 is a perspective view illustrating a skin care device according to an embodiment of the present disclosure. FIG. 2 is a perspective view illustrating a package including a skin care device and a cradle illustrated in FIG. 1.

Referring to FIGS. 1 and 2, a skin care device 1 according to an embodiment of the present disclosure may be a cleanser type device that cleans the skin by contacting the user's skin. The skin care device 1 may be implemented as a portable skin cleanser that can be used without an external power connection by having a battery therein. In this case, the skin care device 1 may be mounted on the cradle 4 during storage or charging.

The skin care device 1 may include a main body 2 and a head 3.

The main body 2 may have a shape in which the user can easily clean the skin by holding the main body by hand and being the ultrasonic vibrator assembly 311 and the brush 322 of the head 3 in close contact with the skin. As an example, at least one surface of the main body 2 is formed to be rounded, so that the user can easily grasp the main body 2 by hand.

A receiving space for receiving various components (circuits, chips, batteries, or the like) may be formed inside the main body 2, and the cover 201 is formed to surround the receiving space, so that the parts inside the receiving space can be protected.

According to an embodiment, the cover 201 may be implemented with a material for preventing moisture, such as water, from permeating into the receiving space. For example, the cover 201 may be implemented as a cover made of a silicon material, but is not limited thereto.

In addition, at least one button 202, 203 for user manipulation is provided on one surface of the main body 2, and at least one indicator 204, 205 for notifying the operating state or battery state of the skin care device 1 may be provided.

For example, at least one button 202, 203 may include a first button 202 for turning on/off the power of the skin care device 1 and a second button 203 for changing an operation mode (vibration intensity or the like) of the skin care device 1.

The at least one indicator 204, 205 may be formed at a position corresponding to the at least one light source provided inside the main body 2 to transmit light emitted from the light source to the outside. For example, at least one indicator 204, 205 may include a first indicator 204 for notifying whether the skin care device 1 is powered on/off or information related to a currently set operation mode, and a second indicator 205 for notifying information related to the state of a battery of the skin care device 1.

The head 3 may be formed on a portion of one surface (for example, a front surface) of the main body. The head 3 forms a contact surface with the skin, so that a predetermined physical stimulus can be applied to the skin. For example, the head 3 may include an ultrasonic vibrator assembly 311 that applies ultrasonic vibrations to the skin, and a brush 322 that applies micro-vibrations. For example, as illustrated in FIG. 1, the brush 322 may be implemented in a ring or donut shape surrounding the outside of the ultrasonic vibrator assembly 311, but this is not necessarily the case.

Hereinafter, components included in the skin care device 1 will be described in more detail with reference to FIG. 3.

FIG. 3 is an exploded view illustrating the skin care device illustrated in FIG. 1.

In the following drawings, the direction in which the ultrasonic vibrator assembly 311 and the brush 322 face is defined as a forward direction, the portion where the head 3 is disposed is defined as the upper portion, and the portion where the speaker assembly 25 is disposed is defined as the lower portion.

Referring to FIG. 3, the main body 2 may include a cover 201, a front case 21, a rear case 22, a substrate 23, a battery 24, a speaker assembly 25, a speaker cover assembly 26, a sealing member 27, and the like.

The cover 201 may be formed to surround at least a portion of the front case 21 and the rear case 22. The inner surface of the cover 201 may be in close contact with the outer surfaces of the front case 21 and the rear case 22. As described above, the cover 201 may be implemented with a material such as silicon to prevent moisture from permeating into the main body 2.

The front case 21 may form a front surface of the main body 2, and the rear case 22 may form a rear surface of the main body 2. The front case 21 and the rear case 22 may be fastened to each other through a plurality of fastening members (for example, screws). As the front case 21 and the rear case 22 are fastened, a receiving space in which the components such as the substrate 23, the battery 24, and the speaker assembly 25 are received may be formed inside the front case 21 and the rear case 22. The front case 21 and the rear case 22 may be implemented with a material such as plastic.

In addition, some components of the head 3 may be received in the receiving space formed by the front case 21 and the rear case 22. For example, a part of the ultrasonic vibrator assembly 311, the bracket 31, and the vibration motor 32 may be received in the receiving space.

An opening through which a portion of the ultrasonic vibrator assembly 311 passes may be formed in the front case 21. A part of the ultrasonic vibrator assembly 311 received in the receiving space may be exposed to the outside through the opening to form a contact surface with the skin.

Meanwhile, a region of the outer surface of the front case 21 which is adjacent to the opening (or surrounding the opening) may form a mounting region of the brush bracket 321.

In addition, at least one opening may be further formed in the front case 21 at a position corresponding to at least one button and/or at least one light source provided on the substrate 23.

At least one component included in the speaker assembly 25 may be fastened to the rear case 22. According to an embodiment, a space corresponding to a resonator of the speaker assembly 25 may be formed in the rear case 22. A speaker hole for emitting the sound generated by the speaker assembly 25 to the outside is formed in the lower side of the rear case 22, and the speaker cover assembly 26 may be mounted in the speaker hole. The speaker cover assembly 26 may be provided with a moisture-permeable and waterproof membrane for preventing water or the like from permeating through the speaker hole.

According to an embodiment, at least one power connection terminal 241 may be further formed on the lower side of the rear case 22. The power connection terminal 241 may be electrically connected to the battery 24. An opening may be formed in a region of the cover 201 corresponding to the power connection terminal 241, and the power connection terminal 241 may be exposed to the outside through the opening. When the skin care device 1 is mounted on the cradle 4, the power connection terminal 241 may be in contact with a power supply terminal (not illustrated) provided in the cradle 4 to receive power from the outside. The supplied power is provided to the battery 24 so that the battery 24 can be charged.

The substrate 23 may be received in a receiving space between the front case 21 and the rear case 22. The substrate 23 may be fastened and fixed to at least one of the front case 21 and the rear case 22. The substrate 23 may be provided with various control configurations related to the operation of the skin care device 1. For example, the control components may include a processor, a memory, a communication circuit (communication interface), an input interface (button or the like), an output interface (light source or the like.), or the like. The processor may be connected to the speaker assembly 25, the ultrasonic vibrator assembly 311, and the vibration motor 32 to control respective operations.

A battery 24 may be provided at the rear of the substrate 23. The battery 24 may be mounted and fixed to the rear case 22 or the rear surface of the substrate 23. The battery 24 may supply power for the operation of the skin care device 1 to each component. As described above, as the skin care device 1 is mounted on the cradle 4, the battery 24 may receive power for charging from the outside through the power connection terminal 241.

According to an embodiment, the skin care device 1 may be connected to an external power supply means to apply a current to the ultrasonic vibrator assembly 311 or drive the vibration motor 32 using power provided from the outside. In this case, the skin care device 1 may not be provided with the battery 24 but may only be provided with means such as a capacitor.

According to an embodiment, a sealing member 27 may be provided between the front case 21 and the rear case 22. For example, an edge region of each of the front case 21 and the rear case 22 may form a contact area during fastening. As illustrated, the contact area may correspond to a region such as a closed curved shape (for example, an ellipse), and the sealing member 27 may be implemented in a closed curved ring shape corresponding to the contact area.

The sealing member 27 seals a gap generated in the contact area when the front case 21 and the rear case 22 are fastened, thereby preventing moisture from permeating into the inside through the gap.

With continued reference to FIG. 3, the head 3 may include a bracket 31, an ultrasonic vibrator assembly 311, a vibration motor 32, a brush bracket 321, and a brush 322.

The bracket 31 may be fastened to the front case 21 and/or the rear case 22 to be received in a receiving space between the front case 21 and the rear case 22.

The ultrasonic vibrator assembly 311 may be mounted on the front of the bracket 31, and the vibration motor 32 may be mounted on the rear side of the bracket 31.

The ultrasonic vibrator assembly 311 may have a cylindrical shape having a predetermined height. The bottom surface of the ultrasonic vibrator assembly 311 may be mounted on the bracket 31 to be located in the receiving space, and the upper surface of the ultrasonic vibrator assembly 311 may be exposed to the outside through the opening of the front case 21 to form a contact surface with the skin. According to an embodiment, at least one sealing ring 313 is formed between the ultrasonic vibrator assembly 311 and the front case 21, so that it is possible to prevent moisture or the like from permeating into the interior through the gap between the ultrasonic vibrator assembly 311 and the front case 21.

The ultrasonic vibrator assembly 311 may generate ultrasonic vibrations based on a current applied under the control of the processor. The ultrasonic vibration creates temporary cracks in the stratum corneum of the skin, so that micro dust or contaminants on the skin surface can be discharged to the outside of the skin, and the removal rate of dead skin cells present on the skin surface can be improved.

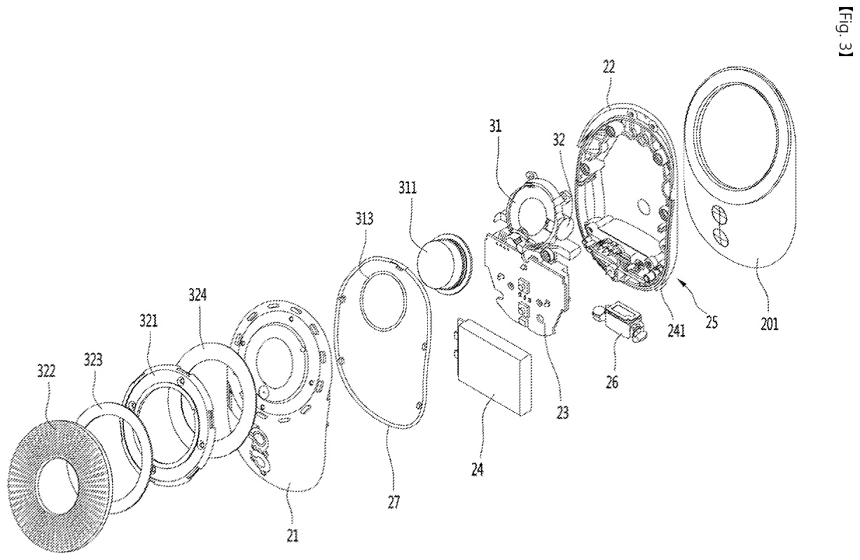

Meanwhile, ultrasonic waves can be classified as providing functions such as exfoliation, skin massage, image acquisition inside the human body, and skin tissue removal according to characteristics. According to an embodiment of the present disclosure, the ultrasonic vibrator assembly 311 may provide ultrasonic vibration having a characteristic of maximizing the cleaning power of wastes or contaminants on the skin surface. Specific details related thereto will be described later with reference to FIGS. 4 to 17.

The vibration motor 32 may be driven under the control of the processor. As the vibration motor 32 is driven, the skin care device 1 may vibrate (vibrate finely) in the front and rear direction. In this case, micro-vibrations may be transmitted to the skin through the brush 322 in contact with the skin. When the micro-vibration is transmitted to the skin through the brush 322, the amount of foam generated by the cleaning agent applied to the skin surface (for example, cleansing foam or the like) increases, so that the cleaning power for contaminants, cosmetics, or the like present on the skin surface can be improved.

The brush bracket 321 may be formed in a ring shape. The brush bracket 321 may be fastened (mounted or attached) to a region of the outer surfaces of the front case 21 surrounding the opening through which the upper surface of the ultrasonic vibrator assembly 311 passes.

A brush 322 may be fastened (mounted or attached) to the front of the brush bracket 321. The brush 322 may include protrusions made of a silicone material that is harmless to the human body. An opening through which the upper surface of the ultrasonic vibrator assembly 311 passes is formed on the center of the brush 322, so that the upper surface of the ultrasonic vibrator assembly 311 is exposed to the outside through the opening to be in contact with the skin.

The brush 322 may stimulate the skin by vibrating according to the driving of the vibration motor 32. By vibration of the brush 322, the amount of foaming of the cleaning agent applied to the skin surface may be increased, and contaminants adhering to the skin surface may be effectively separated from the skin. Accordingly, it may be possible to effectively clean the skin.

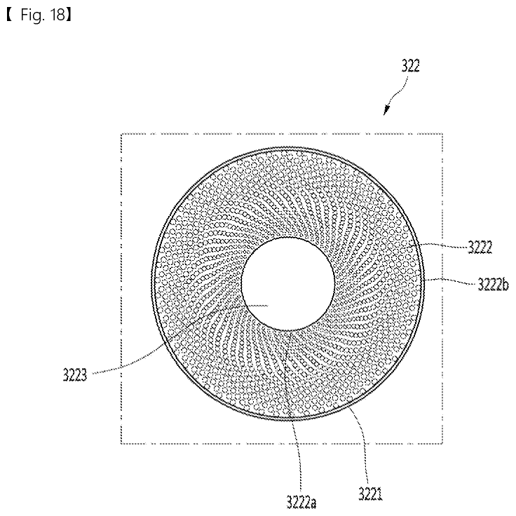

Meanwhile, according to an embodiment of the present disclosure, the protrusions of the brush 322 may have a pattern, thickness, hardness, or the like for maximizing cleaning power on the skin. Specific details related thereto will be described later with reference to FIGS. 18 to 34.

According to an embodiment, the brush 322 may be attached to the brush bracket 321 through the adhesive member 323, and the brush bracket 321 may also be attached to the front case 21 through the adhesive member 324. For example, the adhesive member 323 may include various types of adhesives such as double-sided tape.

FIG. 4 is a cross-sectional view for explaining the structure of the ultrasonic vibrator assembly illustrated in FIG. 3.

Referring to FIG. 4, the ultrasonic vibrator assembly 311 may include an ultrasonic vibrator case 3111, a vibrator 3112, an insulating film 3113, and electrodes 3114 and 3115.

The ultrasonic vibrator case 3111 may be implemented with a metal such as stainless steel having conductivity.

A receiving space S in which the vibrator 3112 is received may be formed in the ultrasonic vibrator case 3111. At least a partial region of one surface (for example, lower surface) of the ultrasonic vibrator case 3111 is opened, and the vibrator 3112 may be inserted and assembled into the receiving space S through the open region. The lower surface of the ultrasonic vibrator case 3111 may be fastened to the bracket 31 described above in FIG. 3, and accordingly, the ultrasonic vibrator assembly 311 may be fixed to the main body 2.

One surface (for example, an upper surface) of the ultrasonic vibrator case 3111 may form a contact surface 311a with the skin.

At least one of a height and a thickness of the ultrasonic vibrator case 3111 may vary according to a thickness of the vibrator 3112. For example, when the thickness of the vibrator 3112 is reduced, at least one of the height and the thickness of the ultrasonic vibrator case 3111 may be reduced, and when the thickness of the vibrator 3112 becomes thicker, at least one of the height and thickness of the ultrasonic vibrator case 3111 may increase.

The vibrator 3112 may be received in the receiving space S of the ultrasonic vibrator case 3111. The vibrator 3112 may be made of ceramic, but is not limited thereto. An insulating film 3113 may be disposed between the ultrasonic vibrator case 3111 and the vibrator 3112. The insulating film 3113 may be made of polyimide, but is not limited thereto. The insulating film 3113 may electrically insulate the ultrasonic vibrator case 3111 and the vibrator 3112, thereby blocking current applied to the vibrator 3112 from flowing to the ultrasonic vibrator case 3111.

The vibrator 3112 is electrically connected to the substrate 23 and the battery 24 through the first electrode 3114 and the second electrode 3115 and may vibrate ultrasonically based on a voltage applied through the first electrode 3114 and the second electrode 3115.

The first electrode 3114 and the second electrode 3115 may be connected to different surfaces of the vibrator 3112. According to an embodiment, an electrode (for example, the second electrode 3115) connected to a surface facing the insulating film 3113 among both surfaces of the vibrator 3112 may extend to a portion of the surface connected to the first electrode 3114 through the side surface of the vibrator 3112 for easy wiring connection. In this case, the insulating portion 3116 may be formed in the remaining region of the contact area between the second electrode 3115 and the vibrator 3112, except for the surface facing the insulating film 3113.

A processor (not illustrated) formed on the substrate 23 may apply a voltage for ultrasonic vibration to the vibrator 3112 in a mode in which ultrasonic vibration is provided.

Meanwhile, in the conventional case, ultrasonic waves (or ultrasonic vibration) have been used to obtain an image inside the human body, provide a massage function through low-frequency vibration, or provide a function of improving the permeation of active ingredients into the skin. Optimal ultrasonic characteristics (frequency, output, or the like) for providing the above functions may be different from each other.

According to an embodiment of the present disclosure, the ultrasonic vibration provided to the skin through the ultrasonic vibrator assembly 311 is for cleaning wastes or contaminants existing on the skin surface and may have ultrasonic vibration characteristics different from other conventional functions.

Specifically, when ultrasonic vibration is propagated to the cleaning agent (cleansing solution) applied to the skin, bubbles are generated in the cleaning agent according to the cavitation effect, and the process of expanding and exploding the bubbles may be repeated. A gap may be formed between the contaminants by the force applied to the contaminants during the expansion and explosion of the air bubbles, and the contaminants may be separated from the skin as the air bubbles permeate and explode through the gaps. In other words, the contaminants on the skin surface can be removed from the skin by being dispersed and decomposed by the pressure according to the expansion and explosion of the air bubbles.

In other words, the skin care device 1 according to the present disclosure may be implemented to have ultrasonic vibration characteristics for maximizing cleaning power. Hereinafter, with reference to FIGS. 5 to 16, the characteristics of ultrasonic vibration set in the skin care device 1 of the present disclosure will be described through various experimental data performed to explore the characteristics of ultrasonic vibration for maximizing cleaning power.

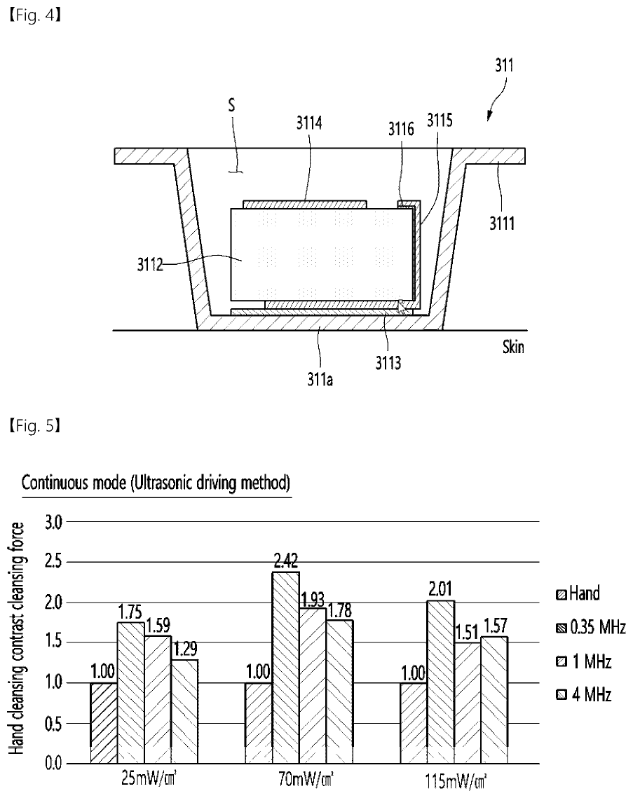

FIG. 5 is an example of experimental data obtained by measuring the difference in cleaning power according to a change in the frequency and output of ultrasonic vibration. FIGS. 6 to 8 are examples of experimental data obtained by measuring the difference in the cleansing region, the difference in the residual region of the waste mimetic body, and the difference in skin brightness according to the change in the frequency and output of ultrasonic vibration.

The experimental data of FIG. 5 illustrates the cleaning power compared to hand cleansing when the frequency of ultrasonic vibration is set to 0.35 MHz, 1 MHz, and 4 MHz, and the output of ultrasonic vibration is set to 25 mW/cm², 70 mW/cm², and 115 mW/cm².

Referring to FIG. 5, since, when the frequency of ultrasonic vibration is 0.35 MHz and the output of ultrasonic vibration is 70 mW/cm², the cleaning power is 2.42 times that of hand cleansing, it can be seen that it is higher than other frequencies or outputs.

Meanwhile, it can be seen that even under conditions other than the above conditions, the cleaning power is 1.29 times to 2.01 times higher than that of hand cleansing. In other words, when the frequency of ultrasonic vibration is within 4 MHz and the output is 255 mW/cm² to 115 mW/cm², superior cleaning power may be provided compared to hand cleansing.

FIGS. 6 to 8 illustrate data of experiments in which a cleaning agent is applied after applying a waste mimetic body to a predetermined region of the skin, and the frequency and output of ultrasonic vibration are set differently to perform cleansing on the predetermined region.

Referring to FIG. 6, similar to the experimental data of FIG. 5, since, when the frequency of ultrasonic vibration is 0.35 MHz and the output of ultrasonic vibration is 70 mW/cm², the size of the cleansing region (the region from which the waste mimetic body is removed) is 3.25 times that of hand cleansing, and it can be seen that the size of the cleansing region is the largest compared to the frequency or output.

In addition, referring to FIG. 7, since, when the frequency of ultrasonic vibration is 0.35 MHz and the output is 70 mW/cm², the size of the residual region of the waste mimetic body is 5.52 times smaller than that of hand cleansing, it can be confirmed that the size of the residual region of the waste mimetic body is the smallest compared to other frequencies or outputs.

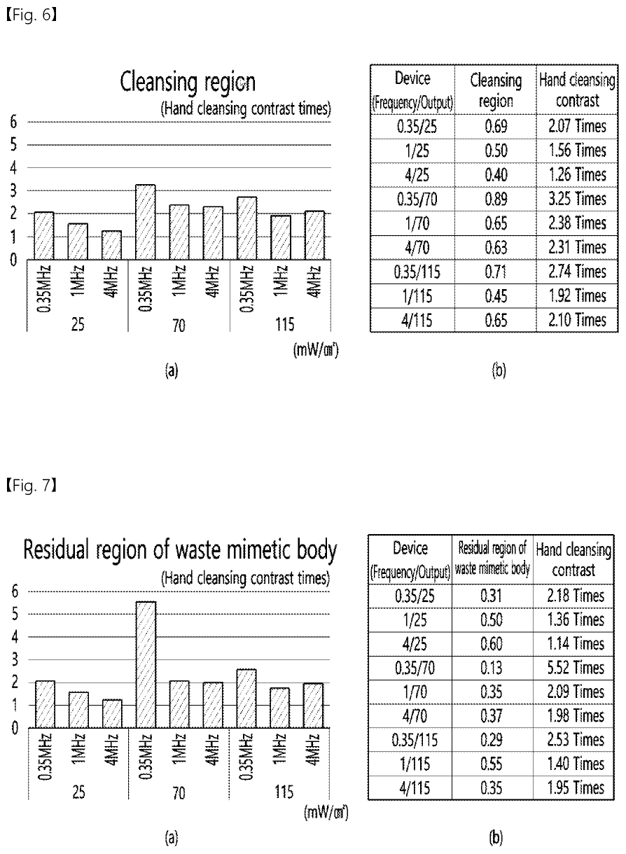

In addition, referring to FIG. 8, since, when the frequency of ultrasonic vibration is 0.35 MHz and the output of ultrasonic vibration is 70 mW/cm², the difference in skin brightness (the difference between the brightness of the region where the waste mimetic body is not applied and the brightness after cleansing of the waste mimetic body region) is 2.68 times smaller than hand cleansing, it can be seen that waste is most effectively removed compared to other frequencies or outputs.

In other words, according to the experimental data of FIGS. 5 to 8, when the frequency of ultrasonic vibration applied to the skin is close to 0.35 MHz, excellent cleaning power can be provided. Based on this, the frequency range of the ultrasonic vibration applied to the skin from the ultrasonic vibrator assembly 311 according to the embodiment of the present disclosure may be set to include 0.35 MHz. For example, the ultrasonic vibration frequency range of the ultrasonic vibrator assembly 311 may be set in the range of 0.3 MHz to 0.4 MHz.

In addition, when the output of ultrasonic vibration applied to the skin is close to 70 mW/cm², excellent cleaning power may be provided. Based on this, the output range of the ultrasonic vibration applied to the skin from the ultrasonic vibrator assembly 311 according to the embodiment of the present disclosure may include 70 mW/cm². For example, the ultrasonic vibration output range of the ultrasonic vibrator assembly 311 may be set in the range of 30 mW/cm² to 110 mW/cm².

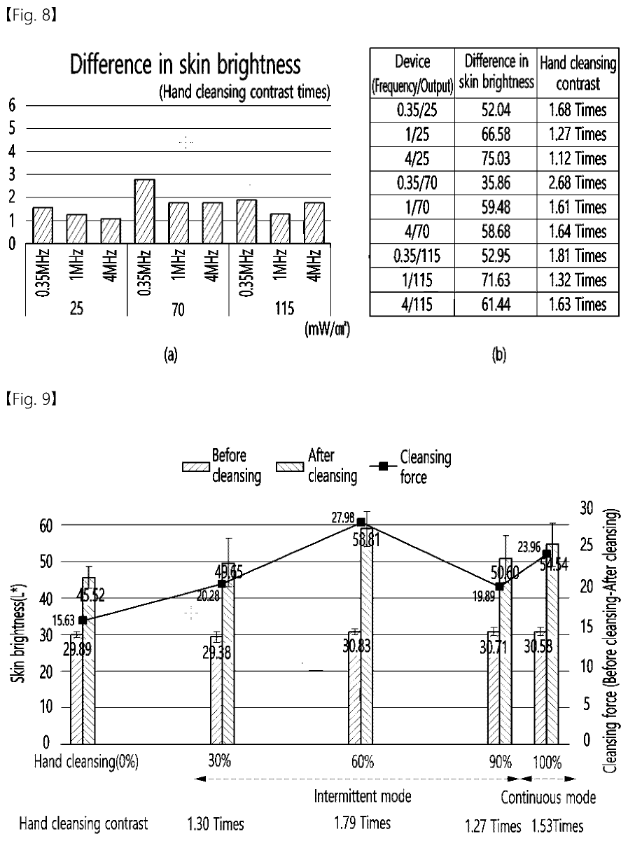

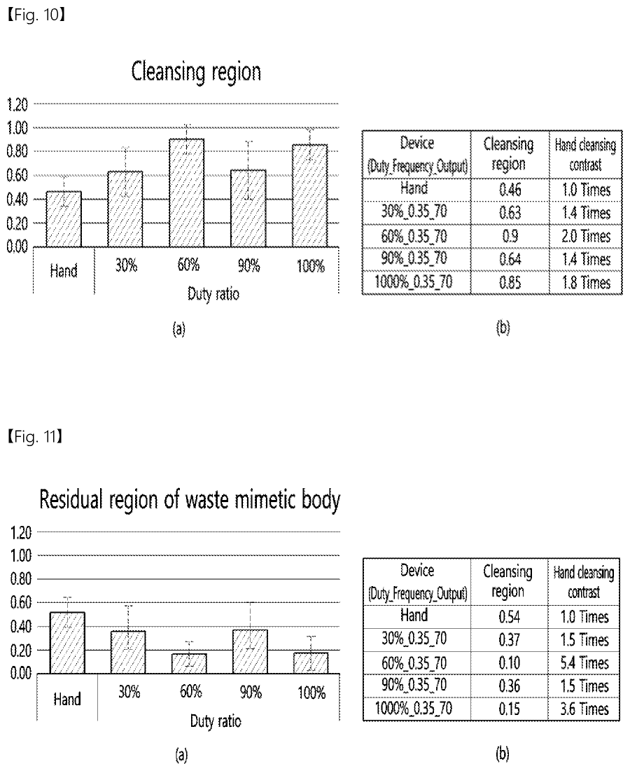

FIG. 9 is an example of experimental data obtained by measuring a difference in cleaning power according to a change in a duty ratio of ultrasonic vibrations. FIGS. 10 to 12 are examples of experimental data obtained by measuring the difference in the cleansing region, the difference in the residual region of the waste mimetic body, and the difference in skin brightness according to the change in the duty ratio of ultrasonic vibration.

The experimental data of FIG. 9 illustrates skin brightness changes (changes in brightness before and after cleaning) compared to hand cleansing when the duty ratio is set to 30%, 60%, and 90% intermittent mode when ultrasonic vibration is applied, and when the duty ratio is set to 100% continuous mode.

Referring to FIG. 9, when the duty ratio of ultrasonic vibration is 60%, it can be seen that the difference in skin brightness before and after washing is the largest, and it is about 1.79 times that of hand cleansing. On the other hand, when the duty ratio is 30% or 90%, it can be seen that the cleaning effect is not significantly greater than that of hand cleansing.

FIGS. 10 to 12 illustrate data of experiments in which a cleaning agent is applied after applying a waste mimetic body to a predetermined region of the skin, and the duty ratio of ultrasonic vibration is set to be different from each other to perform cleansing on the predetermined region.

Referring to FIG. 10, similarly to the experimental data of FIG. 9, since, when the duty ratio of ultrasonic vibration is 60%, the size of the cleansing region (the region from which the waste mimetic body is removed) is about twice that of hand cleansing, it can be seen that the size of the cleansing region is the largest, compared to other duty ratios.

In addition, referring to FIG. 11, since, when the duty ratio of the ultrasonic vibration is 60%, the size of the residual region of the waste mimetic body is about 5.4 times smaller than that of hand cleansing, it can be seen that the size of the residual region of the waste mimetic body is the smallest compared to other duty ratios.

In addition, referring to FIG. 12, since, when the duty ratio of ultrasonic vibration is 60%, the difference in skin brightness (the difference between the brightness of the region where the waste mimetic body is not applied and the brightness after cleansing of the waste mimetic body region) is about 2.4 times that of hand cleansing, it can be seen that wastes are most effectively removed compared to other duty ratios.

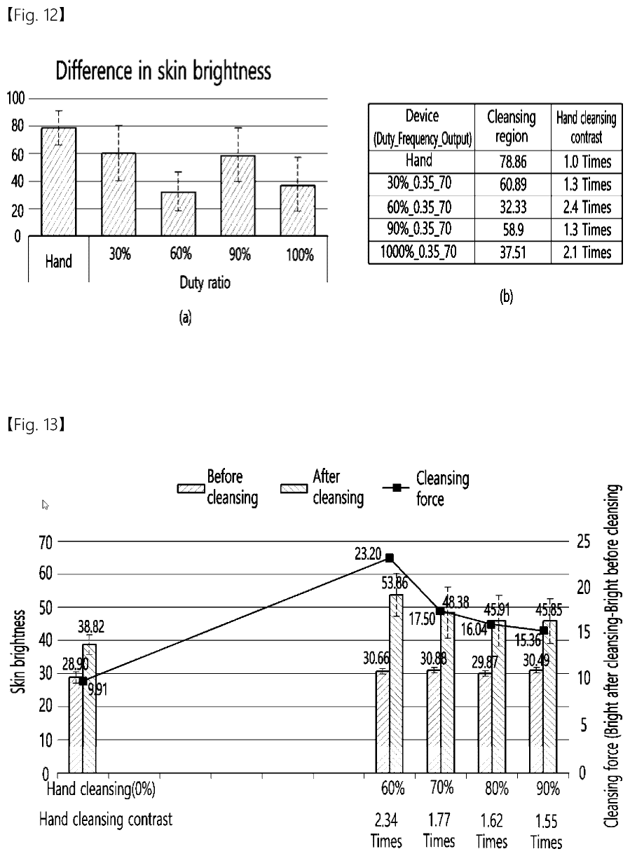

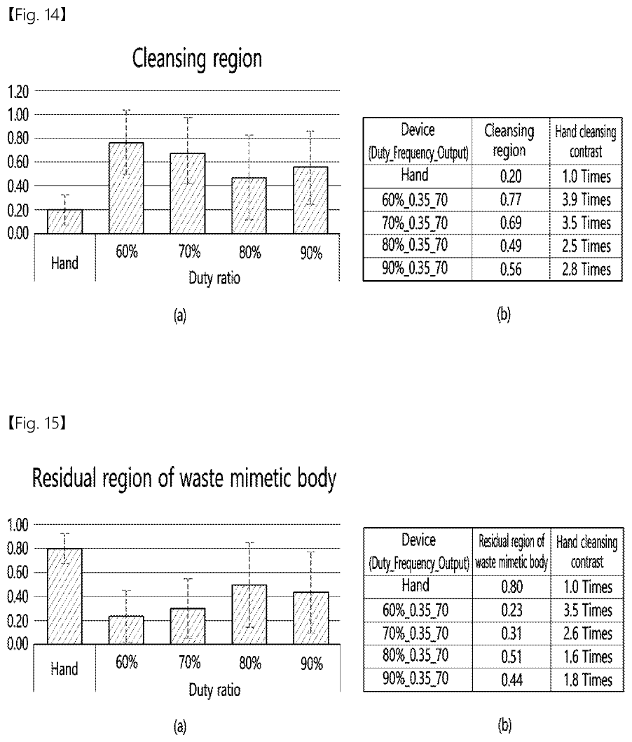

FIG. 13 is an example of experimental data obtained by measuring a difference in cleaning power according to a change in a duty ratio in an intermittent mode of ultrasonic vibration. FIGS. 14 to 16 are examples of experimental data obtained by measuring the difference in the cleansing region, the difference in the residual region of the waste mimetic body, and the difference in skin brightness according to the change of the duty ratio in the intermittent mode of ultrasonic vibration.

In the experimental data of FIGS. 13 to 16, the cleaning power was measured by further subdividing the duty ratio of the intermittent mode (60%, 70%, 80%, 90%).

Referring to FIG. 13, it can be seen that the cleaning power when the duty ratio of the ultrasonic vibration is 60% is significantly higher than the cleaning power when the duty ratio is 70%, 80%, and 90%.

Referring to FIG. 14, similar to the experimental data of FIG. 13, since, when the duty ratio of ultrasonic vibration is 60%, the size of the cleansing region (the region from which the waste mimetic body is removed) is about 3.9 times that of hand cleansing, it can be seen that the size of the cleansing area is the largest compared to other duty ratios. Meanwhile, since, when the duty ratio is 70%, the size of the cleansing region is about 3.5 times that of hand cleansing, it can be seen that the difference from when the duty ratio is 80% or 90% is significant.

In addition, referring to FIG. 15, when the duty ratio of ultrasonic vibration is 60%, the size of the residual region of the waste mimetic body is about 3.5 times smaller than that of hand cleansing, it can be seen that the size of the residual region of the waste mimetic body is the smallest compared to other duty ratios. Meanwhile, since, when the duty ratio is 70%, the size of the residual region of the waste mimetic body is about 2.6 times smaller than that of hand cleansing, and it can be seen that the difference from when the duty ratio is 80% or 90% is significant.

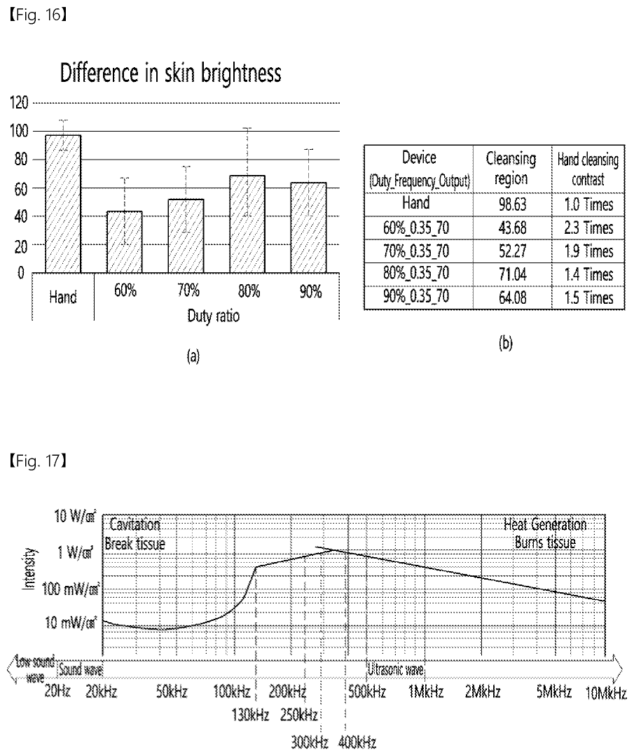

Referring to FIG. 16, since, when the duty ratio of ultrasonic vibration is 60%, the difference in skin brightness (the difference between the brightness of the region where the waste mimetic body is not applied and the brightness after cleansing of the waste mimetic body region) is about 2.3 times smaller than that of hand cleansing, It can be seen that wastes are most effectively removed compared to other duty ratios. Meanwhile, since the difference in skin brightness when the duty ratio is 70% is about 1.9 times smaller than that of hand cleansing, it can be seen that the difference from when the duty ratio is 80% or 90% is significant.

In other words, according to the experimental data of FIGS. 9 to 16, when the duty ratio of ultrasonic vibration applied to the skin is close to 60%, excellent cleaning power can be provided, and even at a duty ratio of 70%, it can provide a significant difference in cleaning power from other duty ratios. Based on this, the duty ratio range of the ultrasonic vibration applied to the skin from the ultrasonic vibrator assembly 311 according to the embodiment of the present disclosure may be set to include 60%. For example, the ultrasonic vibration may be provided in an intermittent mode having a duty ratio ranging from about 50% to about 70%.

FIG. 17 is a diagram illustrating a frequency range of ultrasonic vibration for maximizing cleaning power of a skin care device according to an embodiment of the present disclosure.

Referring to the graph of FIG. 17, the ultrasonic waves may have a threshold value of an intensity safe for the skin which is different for each frequency of ultrasonic waves. For example, when the intensity of ultrasonic waves having a frequency in the range of 20 kHz to 350 kHz exceeds a threshold, there is a risk of damage to skin tissue or cells due to cavitation phenomenon. Also, when the intensity of ultrasonic waves having a frequency of 350 kHz or higher exceeds a threshold, burns may occur.

Meanwhile, ultrasonic waves may be classified for various uses according to frequencies. For example, the use of ultrasonic waves may be divided into treatment (tissue removal, drug delivery, or the like), diagnosis (acquisition of images inside the human body), and skin beauty (exfoliation, absorption promotion, lifting, or the like). Accordingly, the frequency and output of the device that applies the ultrasonic wave may be set differently depending on the purpose.

In the case of a device used for treatment (drug delivery or tissue removal) or diagnosis (ultrasonic waves image acquisition) in a hospital, or the like, ultrasonic waves may have a relatively high frequency to effectively permeate into the skin. For example, ultrasonic waves generated from a device used for tissue removal may have a frequency of about 1 MHz to 7 MHz. Also, ultrasonic waves generated from a device used for diagnosis (ultrasonic waves image acquisition or the like) may have a high frequency of about 2 MHz or more.

Meanwhile, in the case of skin care devices used for skin beauty at home, or the like, since most of the devices are used for exfoliating or lifting on the skin surface, the frequency may be relatively low. For example, the frequency of the device for exfoliation may be set in the range of about 24 KHz to 28 KHz, and the frequency of the lifting (massage) device may be set in the range of about 1 MHz to 3 MHz.

Based on this, the skin care device 1 according to an embodiment of the present disclosure is for removing wastes from the skin surface and may have a lower frequency (less than about 1 MHz) than the frequency of therapeutic or diagnostic ultrasonic waves.

Meanwhile, according to the experimental data of FIGS. 5 to 8, the ultrasonic power of the skin care device 1 may be in the range of 25 mW/cm² to 115 mw/cm², more preferably, it may be set to a value closer to 70 mW/cm² than to 25 mW/cm² and 115 mw/cm².

When the output of the skin care device 1 has the above range, it may be desirable to have a frequency of about 0.13 MHz or more in order to prevent damage to skin tissue or cells.

In addition, based on the experimental data of FIGS. 5 to 8, the ultrasonic frequency of the skin care device 1 may be set closer to 0.35 MHz rather than 0.13 MHz and 1 MHz. For example, the ultrasonic frequency range of the skin care device 1 may have a range of 0.3 MHz to 0.4 MHz.

In addition, based on the experimental data of FIGS. 9 to 16, the ultrasonic waves of the skin care device 1 may be output in an intermittent mode having a duty ratio in the range of 50% to 70%, and more preferably, it may have a duty ratio closer to 60% than the above 50% and 70%.

In other words, the skin care device 1 according to the embodiment of the present disclosure provides ultrasonic vibration according to the frequency, output, and duty ratio set through the experimental data of FIGS. 5 to 16 and thus can maximize the cleaning power of wastes on the skin surface.

FIG. 18 is a view for explaining a brush that vibrates finely by driving a vibration motor illustrated in FIG. 3.

Referring to FIG. 18, the brush 322 may include a base 3221 and a plurality of protrusions 3222 protruding a predetermined height from one surface of the base 3221.

The base 3221 may have a donut shape in which an opening 3223 is formed in a predetermined region including the center. One surface of the base 3221 may form a coupling surface with the brush bracket 321 described above in FIG. 3, and the other surface may be exposed to the front of the skin care device 1 to form a contact surface with the skin.

The plurality of protrusions 3222 may be formed to protrude a predetermined height from the other surface of the base 3221. The plurality of protrusions 3222 may be in contact with the skin to transmit micro-vibrations to the skin surface when the vibration motor 32 is driven.

Meanwhile, the base 3221 and the plurality of protrusions 3222 may be implemented as an integrated structure of a silicon material. Accordingly, it is possible to prevent water or the like from permeating into the skin care device 1 through the brush 322. In addition, since the plurality of protrusions 3222 have ductility, the intensity of stimulation applied to the skin can be easily maintained at a predetermined level or less.

According to an embodiment, the heights of the plurality of protrusions 3222 may be different from each other. Specifically, the height of the first protrusion 3222a formed at a point adjacent to the inner side of the brush 322, that is, the opening 3223, can be lower than the height of the second protrusion 322b formed at a point adjacent to the outside of the brush 322. Accordingly, the user can be effectively close contact the brush 322 with the non-protruding region (for example, the region between the nose and the cheek, or the like) among the skin regions.

Meanwhile, the plurality of protrusions 3222 may have an arrangement pattern, a thickness pattern, and a hardness to maximize the cleaning power of the skin surface. Various experimental data related thereto will be described in detail with reference to FIGS. 19 to 34.

Experimental data to be described later is the data of the experiment which performs the cleansing of the predetermined region by applying a cleaning agent after applying a waste mimetic body to a predetermined region of the skin, and bringing the brush 322 that microscopically vibrates by driving the vibration motor 32 into contact with the skin.

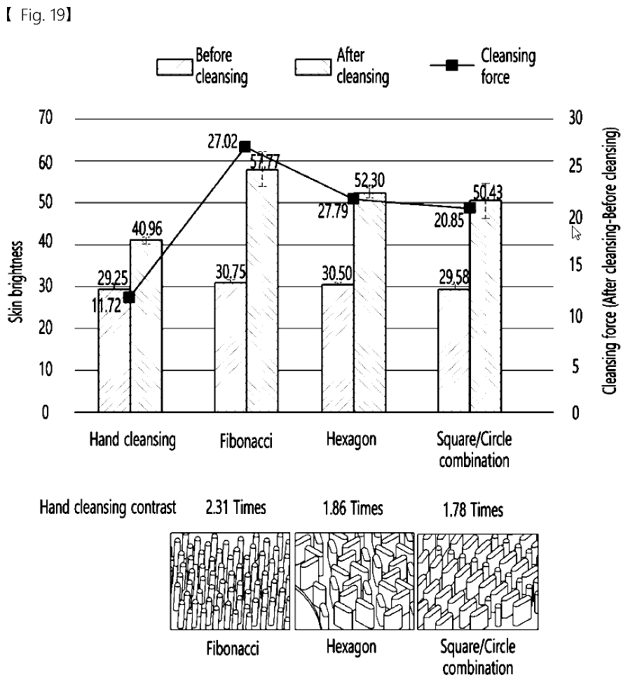

FIG. 19 is an example of experimental data obtained by measuring the difference in cleaning power according to the protrusion shape and pattern of the brush. FIGS. 20 to 22 are examples of experimental data obtained by measuring the difference in the cleansing region, the difference in the residual region of the waste mimetic body, and the difference in skin brightness according to the protrusion shape and pattern of the brush.

Referring to FIG. 19, when the distal ends of the plurality of protrusions 3222 are circular, and the plurality of protrusions 3222 are arranged in a Fibonacci spiral pattern, the cleaning power may correspond to about 2.31 times that of hand cleansing. Meanwhile, when the distal ends of the plurality of protrusions 3222 are oval and arranged in a hexagonal pattern, the cleaning power corresponds to about 1.86 times that of hand cleansing. In addition, when the distal ends of the plurality of protrusions 3222 are a combination of an oval and a circle and are radially arranged, the cleaning power corresponds to about 1.78 times that of hand cleansing.

When the brush 322 has a circular shape (a donut shape), when the plurality of protrusions 3222 are arranged in a Fibonacci spiral pattern, a contact area with the skin can be maximized compared to other types of arrangement. As the contact area with the skin is maximized, the cleaning power may also be higher than that of other types of arrangement.

Referring to FIG. 20, similar to the experimental data of FIG. 19, since, when the distal ends of the plurality of protrusions 3222 are circular and arranged in a Fibonacci spiral pattern, the size of the cleansing region (region from which the waste mimetic body is removed) is about 2.8 times that of hand cleansing, it can be seen that the size of the cleansing region is the largest compared to other protrusion shapes or arrangements.

Also, referring to FIG. 21, since, when the distal ends of the plurality of protrusions 3222 are circular and arranged in a Fibonacci spiral pattern, the size of the residual region of the waste mimetic body is about 6.2 times smaller than that of hand cleansing, it can be seen that the size of the remaining region of the waste mimetic body is the smallest, compared to other protrusion shapes or arrangements.

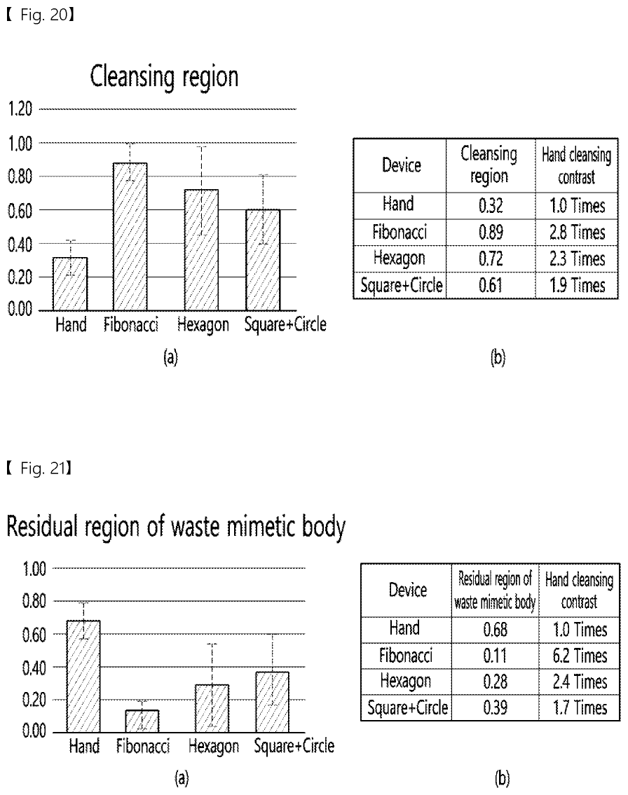

Also, referring to FIG. 22, since, when the distal ends of the plurality of protrusions 3222 are circular and arranged in a Fibonacci spiral pattern, the difference in skin brightness (the difference between the brightness of the region where the waste mimetic body is not applied and the brightness after cleansing of the waste mimetic body region) is about 2.7 times smaller than hand cleansing, it can be seen that wastes are most effectively removed, compared to other protrusion shapes or arrangements.

In other words, based on the experimental data of FIGS. 19 to 22, the plurality of protrusions 3222 formed on the brush 322 may be arranged in a Fibonacci spiral pattern as illustrated in FIG. 18.

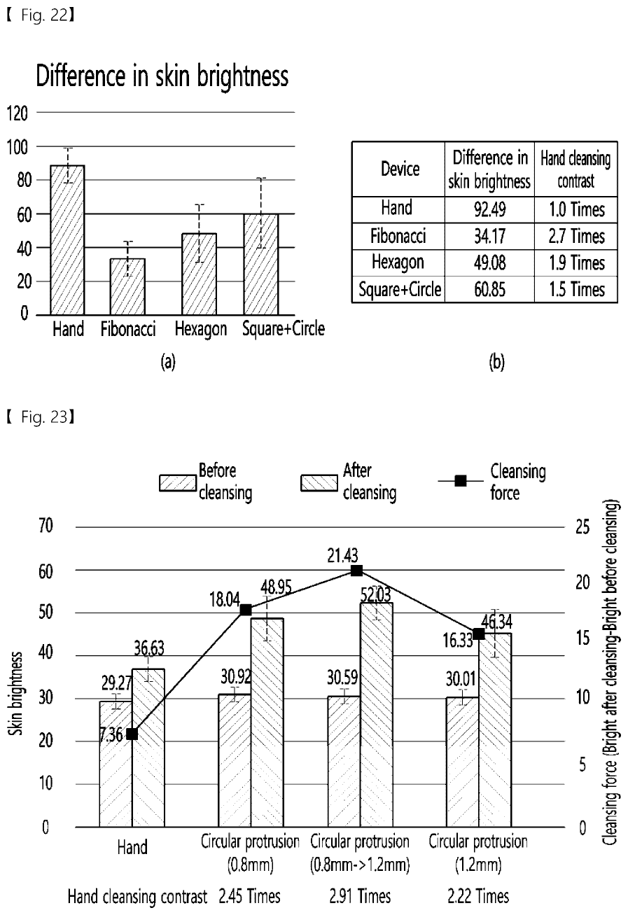

FIG. 23 is an example of experimental data obtained by measuring a difference in cleaning power according to a change in thickness of a plurality of protrusions arranged in a Fibonacci spiral pattern of a brush. FIGS. 24 to 26 are examples of experimental data obtained by measuring the difference in the cleansing region, the difference in the residual region of the waste mimetic body, and the difference in skin brightness according to the change in the thickness of the plurality of protrusions arranged in the Fibonacci spiral pattern of the brush.

According to the experimental data of FIGS. 19 to 22, the plurality of protrusions 3222 may be arranged in a Fibonacci spiral pattern. However, when the thicknesses of the plurality of protrusions 3222 are all constant, the distance between the protrusions increases toward the outside of the brush 322.

The experimental data illustrated in FIGS. 23 to 26 are data of a cleaning power test for a case where the thickness of the protrusions 3222 is constant and a case where the thickness increases toward the outside of the brush 322.

Referring to FIG. 23, it can be seen that the cleaning power when the thickness of the protrusions 3222 is increased toward the outside of the brush 322 is higher than that when the thickness of the protrusions 3222 is uniformly formed. In addition, it can be seen that the cleaning power when the thickness of the protrusions 3222 is 0.8 mm is higher than that when the thickness is 1.2 mm. In other words, it can be seen that the larger the contact area between the protrusions 3222 and the skin, the higher the cleaning power.

Referring to FIG. 24, similar to the experimental data of FIG. 23, since, when the thickness of the plurality of protrusions 3222 increases toward the outside of the brush 322, the size of the cleansing region is about 4.1 times that of hand cleansing, it can be seen that the size of the cleansing region is larger than that in the case of having a constant thickness.

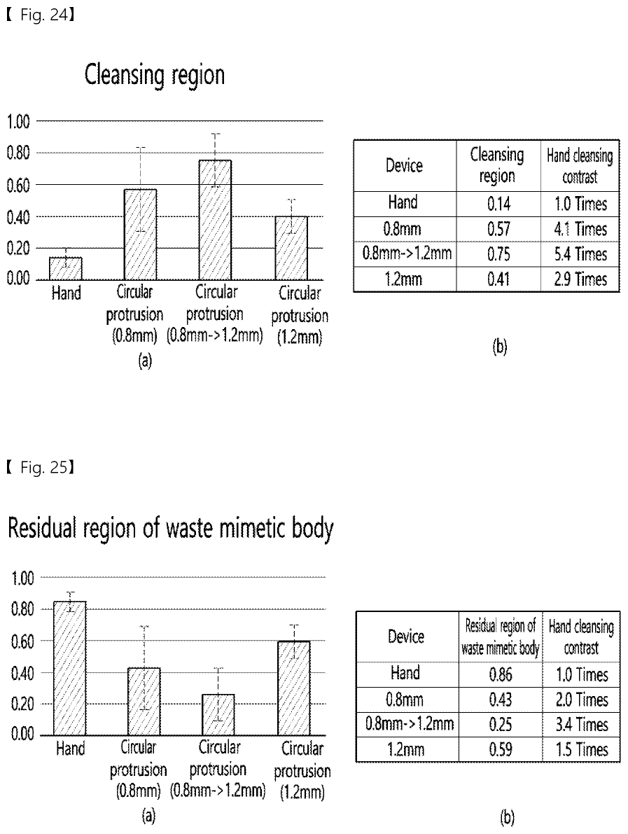

Also, referring to FIG. 25, since, when the thickness of the plurality of protrusions 3222 is formed to increase toward the outside of the brush 322, the size of the residual region of the waste mimetic body is about 3.4 times smaller than that of hand cleansing, It can be seen that the size of the residual region of the waste mimetic body is smaller than that in the case of having a constant thickness.

In addition, referring to FIG. 26, since, when the thickness of the plurality of protrusions 3222 increases toward the outside of the brush 322, the difference in skin brightness (the difference between the brightness of the region where the waste mimetic body is not applied and the brightness after cleansing of the waste mimetic body region) is about 2.2 times smaller than that of hand cleansing, it can be seen that wastes are most effectively removed compared to the case of having a constant thickness.

In other words, based on the experimental data of FIGS. 23 to 26, the thickness of the plurality of protrusions 3222 formed in the Fibonacci spiral pattern on the brush 322 of the present disclosure can increase from the inside to the outside of the brush 322.

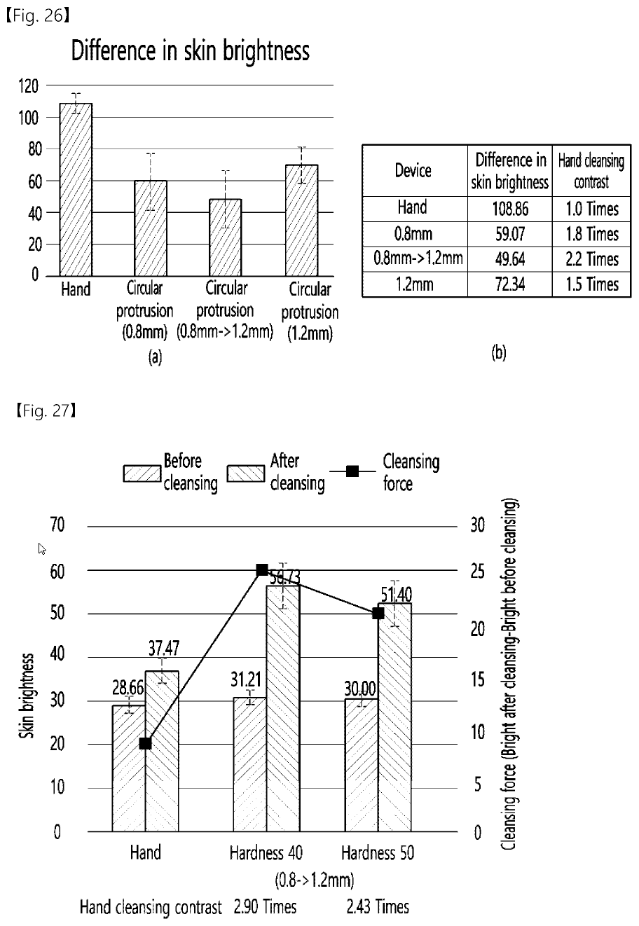

FIG. 27 is an example of experimental data obtained by measuring a difference in cleaning power according to hardness of a plurality of protrusions of a brush. FIGS. 28 to 30 are examples of experimental data obtained by measuring the difference in the cleansing region according to the hardness of the plurality of protrusions of the brush, the difference in the residual region of the waste mimetic body, and the difference in skin brightness.

As described above with reference to FIG. 18, the plurality of protrusions 3222 may be implemented with a silicone material with ductility or the like. At this time, since a difference in cleaning power may occur according to the hardness of the plurality of protrusions 3222, it is necessary to form the plurality of protrusions 3222 with a hardness that can provide the best cleaning power.

The experimental data of FIGS. 27 to 30 is experimental data obtained by comparing the difference in cleaning power according to hardness of the protrusions 3222, when the plurality of protrusions 3222 are arranged in a Fibonacci spiral pattern, and the thickness of the protrusions 3222 is formed thicker toward the outside of the brush 322.

Referring to FIG. 27, it can be seen that the cleaning power when the hardness of the protrusions 3222 is 40 is about 2.9 times that of hand cleansing, which is higher than the cleaning power when the hardness is 50.

Referring to FIG. 28, similar to the experimental data of FIG. 27, it can be seen that the size of the cleansing region when the hardness of the protrusions 3222 is 40 is about 4.4 times that of hand cleansing, which is larger than when the hardness is 50.

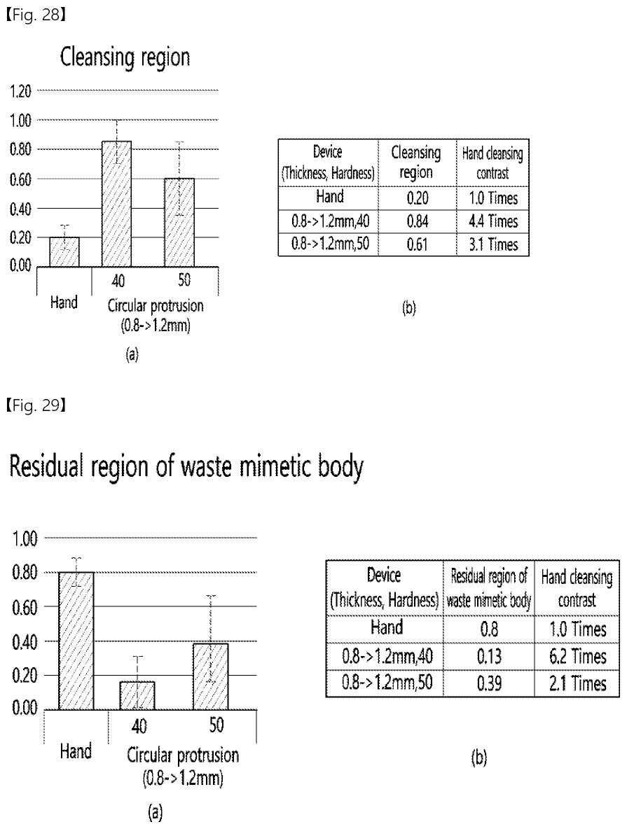

In addition, referring to FIG. 29, since the size of the residual region of the waste mimetic body when the hardness of the protrusions 3222 is 40 is about 6.2 times smaller than that of hand cleansing, it can be seen that the size of the residual area of the waste mimetic body is smaller than that when the hardness is 50.

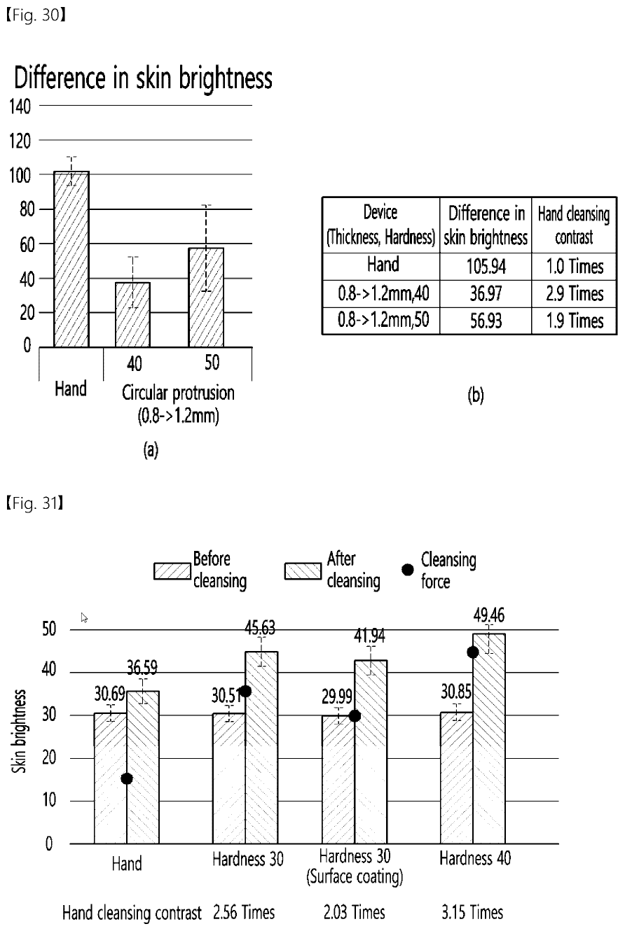

In addition, referring to FIG. 30, since, when the hardness of the protrusions 3222 is 40, the difference in skin brightness (the difference between the brightness of the region where the waste mimetic body is not applied and the brightness after cleansing of the waste mimetic body region) is about 2.9 times smaller than that of hand cleansing, it can be seen that the wastes are most effectively removed compared to when the hardness is 50.

In other words, based on the experimental data of FIGS. 27 to 30, the hardness of the plurality of protrusions 3222 included in the brush 322 of the present disclosure may be closer to 40 than 50.

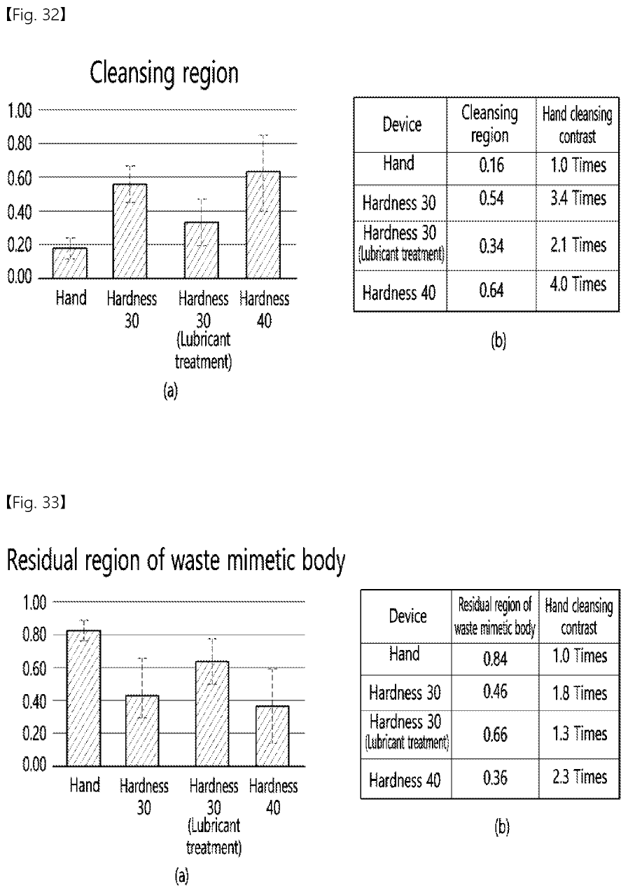

FIG. 31 is an example of experimental data obtained by measuring the difference in cleaning power according to the hardness and surface coating of a plurality of protrusions of the brush. FIGS. 32 to 34 are examples of experimental data obtained by measuring the difference in the cleansing region, the difference in the residual region of the waste mimetic body, and the difference in skin brightness according to the hardness and surface coating of the plurality of protrusions of the brush.

According to the experimental data of FIGS. 27 to 30, it can be seen that the cleaning power when the hardness of the protrusions 3222 is 40 is superior to the cleaning power when the hardness is 50. Hereinafter, in FIGS. 31 to 34, the experimental data obtained by measuring the cleaning power when the hardness of the protrusions 3222 is 30 lower than 40, and the cleaning power when the coating treatment (lubricant treatment or the like) is performed on the surface of the protrusions 3222 is described.

As in FIGS. 27 to 30, in the experiment of FIGS. 31 to 34, the plurality of protrusions 3222 may be arranged in a Fibonacci spiral pattern, and the thickness may increase toward the outside of the brush 322.

Referring to FIG. 31, it can be seen that the cleaning power when the hardness of the protrusions 3222 is 30 is about 2.56 times that of hand cleansing, which is lower than the cleaning power when the hardness is 40. In addition, it can be seen that the cleaning power when the surface of the protrusions 3222 is coated is rather reduced.

Referring to FIG. 32, similar to the experimental data of FIG. 31, since the size of the cleansing region when the hardness of the protrusions 3222 is 40 is about 4 times that of hand cleansing, it can be seen that the size of the cleansing area is larger than when the hardness is 30. In addition, it can be seen that when the coating treatment (lubricant treatment) is performed on the surface of the protrusions 3222, the size of the cleansing region is rather reduced.

In addition, referring to FIG. 33, since, when the hardness of the protrusions 3222 is 40, the size of the residual region of the waste mimetic body is about 2.3 times smaller than that of hand cleansing, it can be seen that the size of the residual region of the waste mimetic body is small, compared to when the hardness is 30. In addition, it can be seen that when the coating treatment (lubricant treatment) is performed on the surface of the protrusions 3222, the size of the residual region of the waste mimetic body is rather increased.

Also, referring to FIG. 34, since, when the hardness of the protrusions 3222 is 40, the difference in skin brightness (the difference between the brightness of the region where the waste mimetic body is not applied and the brightness after cleansing of the waste mimetic body region) is about 1.8 times smaller than that of hand cleansing, it can be seen that wastes are more effectively removed compared to when the hardness is 30. In addition, it can be seen that when the coating treatment (lubricant treatment) is performed on the surface of the protrusions 3222, the cleaning power of wastes is rather reduced.

In other words, based on the experimental data of FIGS. 31 to 34, the hardness of the plurality of protrusions 3222 included in the brush 322 of the present disclosure may be formed closer to 40 than 30, and a separate coating treatment on the surface thereof may not be performed.

Combining the experimental data of FIGS. 27 to 34, the hardness of the plurality of protrusions 3222 of the present disclosure may be formed between 30 and 50. More preferably, the hardness of the plurality of protrusions 3222 is formed closer to 40 rather than 30 and 50, thereby maximizing the cleaning effect.

Based on the experimental data of FIGS. 19 to 34, the plurality of protrusions 3222 included in the brush 322 of the skin care device 1 according to an embodiment of the present disclosure are arranged in a Fibonacci spiral pattern, and the thickness increases toward the outside of the brush 322, and the hardness is formed in a range between about 30 and 50, preferably close to 40, thereby maximizing the cleaning power.

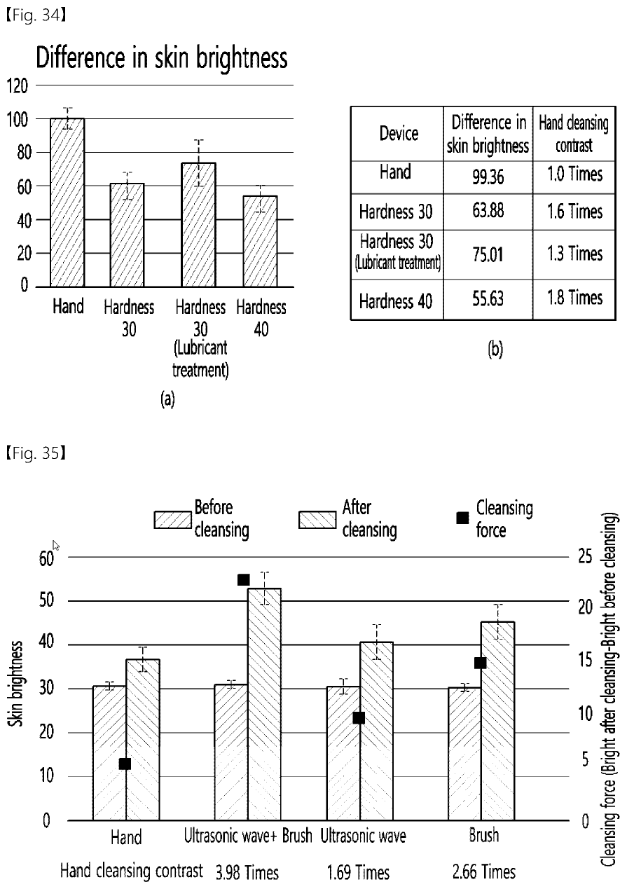

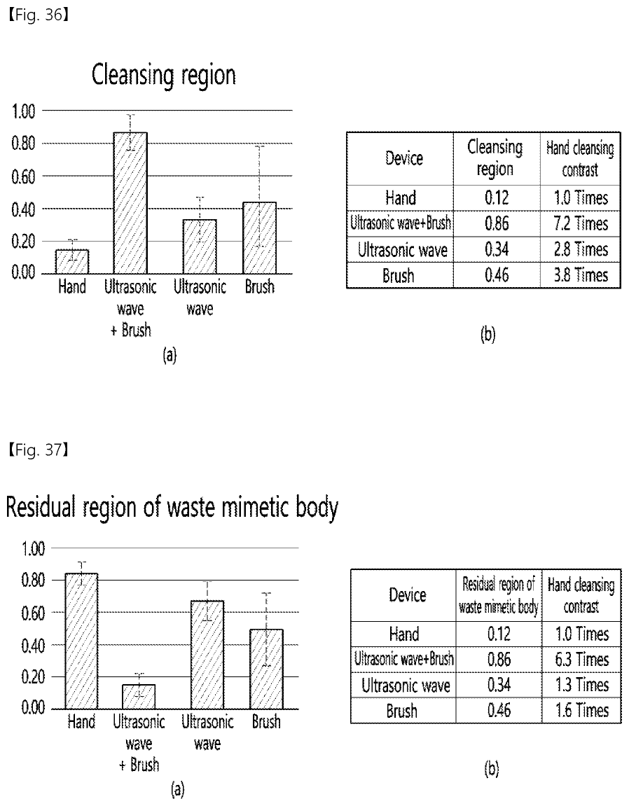

FIG. 35 is an example of experimental data obtained by measuring a difference in cleaning power according to whether a combination of ultrasonic vibration and brush micro-vibration is applied. FIGS. 36 to 38 are experimental data obtained by measuring the difference in the cleansing region, the difference in the residual region of the waste mimetic body, and the difference in skin brightness according to whether the combination of ultrasonic vibration and brush micro-vibration is applied.

In the experiments of FIGS. 35 to 38, ultrasonic vibration characteristics may be set according to the experimental data of FIGS. 9 to 16, and the shape and characteristics of the brush 3222 may be set according to the experimental data of FIGS. 19 to 34.

Based on this, referring to FIG. 35, since, when ultrasonic vibration and brush micro-vibration are applied, the cleaning power is about 3.98 times that of hand cleansing, it can be seen that it provides superior cleaning power compared to the case where only ultrasonic vibration is applied and the case where only brush micro-vibration is applied.

Referring to FIG. 36, similar to the experimental data of FIG. 35, since, when the combination of ultrasonic vibration and brush micro-vibration are applied, the size of the cleansing region is about 7.2 times that of hand cleansing, it can be seen that the size of the cleansing region is large compared to when only ultrasonic vibration is applied and when only brush micro-vibration is applied.

Also, referring to FIG. 37, since, when the combination of ultrasonic vibration and brush micro-vibration are applied, the size of the residual region of the waste mimetic body is about 6.3 times smaller than that of hand cleansing, it can be seen that the size of the residual region of the waste mimetic body is small compared to when only ultrasonic vibration is applied and when only brush micro-vibration is applied.

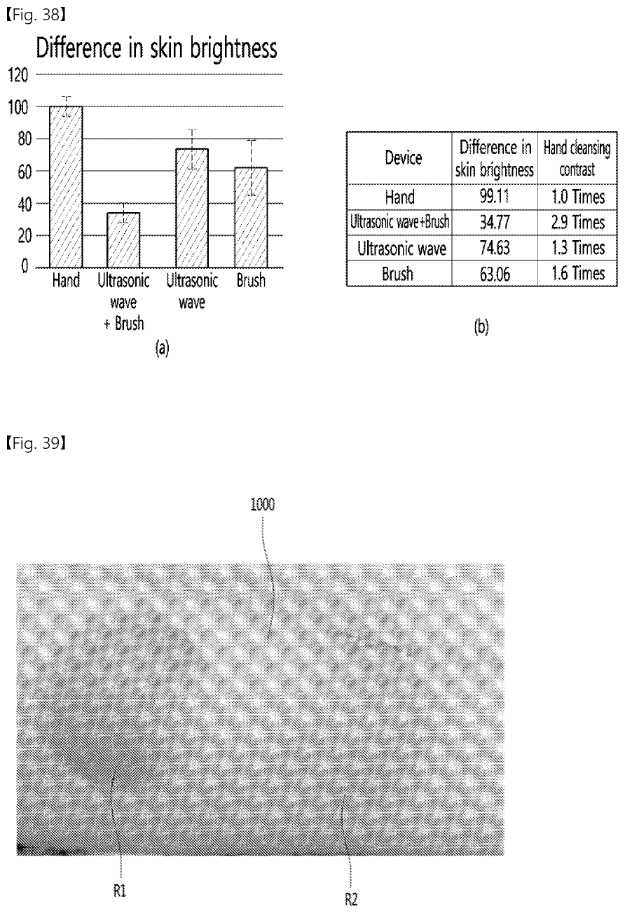

In addition, referring to FIG. 38, since the difference in skin brightness (the difference between the brightness of the region where the waste mimetic body is not applied and the brightness after cleansing of the waste mimetic body region) when ultrasonic vibration and brush micro-vibration are combined is about 2.9 times smaller than that of hand cleansing, it can be seen that wastes are more effectively removed compared to the case where only the ultrasonic vibration is applied and the case where only the brush micro-vibration is applied.

FIG. 39 is an exemplary view illustrating the difference in cleaning power when only one of ultrasonic vibration and brush micro-vibration is applied and when the combination of ultrasonic vibration and brush micro-vibration is applied.

Referring to FIG. 39, cleansing may be performed on the first region R1 and the second region R2 of the user's skin 1000 to which the waste mimetic body is applied. At this time, only one of ultrasonic vibration and brush micro-vibration is applied to the first region R1, and both ultrasonic vibration and brush micro-vibration are applied to the second region R2.

As a result, the difference in skin brightness with the region to which the waste mimetic body is not applied may be smaller in the second region R2 than the first region R1. In other words, it may mean that the cleaning of the second region R2 is more effectively performed.

In other words, based on the experimental data of FIGS. 35 to 38, the skin care device 1 according to an embodiment of the present disclosure includes an ultrasonic vibrator assembly 311 and the brush 322 and thus ultrasonic vibrations and brush micro-vibration may be applied together when cleaning the user's skin. Accordingly, by providing improved cleaning power compared to hand cleansing or other conventional cleansing devices, it is possible to effectively remove wastes present on the skin surface, thereby improving skin health.

The above description is merely illustrative of the technical spirit of the present disclosure, and various modifications and variations will be possible without departing from the essential characteristics of the present disclosure by those skilled in the art to which the present disclosure pertains.

Therefore, the embodiments disclosed in the present disclosure are not intended to limit the technical spirit of the present disclosure, but to explain, and the scope of the technical spirit of the present disclosure is not limited by these embodiments.

The protection scope of the present disclosure should be interpreted by the following claims, and all technical ideas within the scope equivalent thereto should be construed as being included in the scope of the present disclosure.

1. A skin care device comprising:
a main body having a processor; and
an ultrasonic vibrator assembly disposed on one end of the main body and forming a contact surface with a skin of a user,
wherein the processor controls the ultrasonic vibrator assembly to apply an ultrasonic vibration to the skin according to at least one of a frequency characteristic, an output characteristic, and a duty ratio characteristic for removing wastes on a surface of the skin.

2. The skin care device of claim 1, wherein a frequency of the ultrasonic vibration applied to the skin through the ultrasonic vibrator assembly is less than 1 MHz.

3. The skin care device of claim 2, wherein the frequency of the ultrasonic vibration applied to the skin has a range of 0.13 MHz or more and less than 1 MHz.

4. The skin care device of claim 3, wherein the frequency of the ultrasonic vibration applied to the skin is approximately 0.35 MHz.

5. The skin care device of claim 3, wherein the frequency of the ultrasonic vibration applied to the skin has a range of 0.3 MHz or more and 0.4 MHz or less.

6. The skin care device of claim 1, wherein an output of the ultrasonic vibration applied to the skin through the ultrasonic vibrator assembly has a range of approximately 25 mW/cm² or more and less than approximately 115 mW/cm².

7. The skin care device of claim 6, wherein the output of the ultrasonic vibration applied to the skin is approximately 70 mW/cm².

8. The skin care device of claim 1, wherein a duty ratio of the ultrasonic vibration applied to the skin through the ultrasonic vibrator assembly has a range of approximately 50% or more and less than approximately 70%.

9. The skin care device of claim 8, wherein the duty ratio of the ultrasonic vibration applied to the skin is approximately 60%.

10. A skin care device comprising:
a main body having a receiving space for receiving a vibration motor therein; and
a brush disposed at one end of the main body and configured to transmit microvibrations to a skin of a user when the vibration motor is driven,
wherein the brush includes:
a base; and
a plurality of protrusion protruding from one surface of the base and forming a contact surface with the skin, and
wherein the plurality of protrusions are arranged in a Fibonacci spiral pattern on the one surface of the base.

11. The skin care device of claim 10, wherein a distal end of each of the plurality of protrusions is circular.

12. The skin care device of claim 10, wherein a thickness of each of the plurality of protrusions increases from the inside of the base to the outside of the base.

13. The skin care device of claim 10, wherein a height of each of the plurality of protrusions increases from the inside of the base to the outside of the base.

14. The skin care device of claim 10, wherein the plurality of protrusions include a first protrusion, and a second protrusion formed outside the first protrusion, and
wherein a thickness of the first protrusion is thinner than a thickness of the second protrusion.

15. The skin care device of claim 14, wherein a height of the first protrusion is lower than a height of the second protrusion.

16. The skin care device of claim 10, wherein the plurality of protrusions are formed of silicon, and
wherein a hardness of the plurality of protrusions has a range of 30 or more and less than 50.

17. The skin care device of claim 16, wherein the hardness of the plurality of protrusions is approximately 40.

18. A skin care device comprising:
a main body having a receiving space for receiving a processor and a vibration motor formed therein; and
a head formed at one end of the main body,
wherein the head includes:
an ultrasonic vibrator assembly forming a contact surface with a skin of a user, and
a brush including a plurality of protrusions forming another contact surface with the skin of the user.

19. The skin care device of claim 18, wherein the brush further includes a donut-shaped base surrounding an outer circumference of the ultrasonic vibrator assembly,
wherein the plurality of protrusions are arranged in a Fibonacci spiral pattern on one surface of the base,
wherein a thickness of each of the plurality of protrusions increases as a distance from the ultrasonic vibrator assembly increases, and
wherein the plurality of protrusions are formed of silicone having a hardness range of approximately 30 or more and less than approximately 50.

20. The skin care device of claim 18, wherein an ultrasonic vibration applied to the skin through the ultrasonic vibrator assembly has a frequency characteristic of approximately 0.35 MHz, has an output characteristic of approximately 70 mW/cm², and has a duty ratio characteristic of approximately 60%.
